(19) United States
(12) Patent Application Publication
(10) Pub. No.: US 2017/0065199 A1
(43) Pub. Date: Mar. 9, 2017

(54) Monitoring human brain excitability using synchronization measures

(71) Applicant: Christian Meisel, (US)

(72) Inventor: Christian Meisel, (US)

(21) Appl. No.: 14/843,996

(22) Filed: Sep. 3, 2015

(51) Int. Cl. A61B 5/0476; A61B 5/00; A61B 5/16; A61B 5/04

(52) U.S. Cl. CPC A61B 5/0476; A61B 5/04012; A61B 5/4094; A61B 5/165; A61B 5/4818; A61B 5/4815; A61B 5/4848; A61B 5/6803

(57) ABSTRACT

The present invention is directed to a method of continuously monitoring neuronal synchronization in a subject comprising (a) determining a deviation in mean synchronization (R) from a predetermined value at rest, wherein the pre-determined value of R is 1, and the variability of synchronization H; and (b) repeating step (a) one or more times to continuously monitor synchronization R and its variability H in a subject. The invention also features methods of determining and monitoring the degree of brain excitability. The invention furthermore features methods of determining or monitoring the degree of sleep deprivation in a subject, methods of identifying subjects that are susceptible to a sleep disorder and methods of diagnosing a sleep disorder in a subject.

### FIELD OF THE INVENTION

This invention relates generally to the field of brain and brain network excitability in health and disease.

### BACKGROUND OF THE INVENTION

Normal functioning of cortical networks critically depends on a finely tuned level of excitability, the transient or steady-state response in which the brain reacts to a stimulus. The importance of adequate excitabil-ity levels is highlighted by the pathological consequences and impaired performance resulting from aberrant network excitability. In epilepsy, for example, changes in cortical network excitability are believed to be an important cause underlying the initiation and spread of seizures, i.e. the large non-physiological neuronal activity events across time and space. Evidence for changes of excitability in brain networks affected in epilepsy has come from a variety of observations [1, 2, 3, 4, 5, 6, 7]. The insight that epilepsy is related to hyperexcitability is also at the basis of pharmacological treatment options for patients. Most antiepileptic drugs (AED) aim to reduce the excitability in neural tissue by reducing the excitability of individual neurons through selective ion channel blockers, enhancing inhibitory synaptic transmission or inhibiting excitatory synaptic transmission [8].

Apart from aberrant pathological deviations, changes in cortical excitability are believed to play a role in normal conditions during the course of wake and sleep. A study using transcranial magnetic stimulation to study excitability in human cortex found increased responses after a period of sustained wakefulness which was rebalanced after sleep [9, 10]. Such findings suggest that excitability could increase during wake and might result in suboptimal information processing in cortical networks [11, 12] and point to a pivotal role of sleep in rebalancing the level of excitability.

The ability to monitor excitability in brain networks is therefore highly desirable for an understanding of both normal as well as pathological brain function. In epilepsy patients, the ability to monitor excitability and control its degree is of prime importance for adequate clinical care and treatment. To date, reliable measures of cortical excitability based on ongoing activity have been difficult to obtain. Instead, excitability is usually measured as the response to electrical or magnetic stimulation [13, 2, 3, 14]. A disadvantage of these methods, however, is their complex design which limits regular clinical use and continued monitoring of the time course over extended periods of time. Even more so, the fact that such perturbations can induce seizures constitutes a considerable limitation for its application in patients suffering from epilepsy [15]. For these various reasons, methods to monitor brain excitability based on ongoing activity without the need of external perturbations would be highly preferable.

A method to reliably quantify cortical excitability in epilepsy patients would allow to objectively determine the effect of antiepileptic drugs (AED), provide a tool in adjusting AED dosages to optimal levels for a successful treatment on one side and controlling adverse drug effects on the other side. Accordingly, there is a need in the art for new methods for monitoring brain excitability in both health and disease.

### SUMMARY OF THE INVENTION

The present invention provides a robust method to monitor brain activity in order to estimate the excitability in the brain. The present application demonstrates that the synchronization between different brain areas is a valid marker for the excitability of the brain. The present application further demonstrates that this synchronization measure can provide an absolute, objective reference point for normal excitability levels and, consequently, how this method can detect and quantify a deviation from this reference point in epilepsy patients under antiepileptic drug (AED) medication. Specifically, cortex synchronization (R) of normal ongoing brain activity exhibits synchronization values around R˜0.5. The use of antiepileptic drugs bring synchronization in brain networks to lower values (R<0.5) in a dosage dependent manner. The synchronization measure R is therefore demonstrated to be a biomarker for brain excitability with an absolute reference point characterizing normal brain activity (R˜0.5) and, consequently, any deviation therefrom.

Accordingly, the invention monitors brain activity by non-invasive means, e.g. EEG electrodes embedded into a helmet, a scalp EEG system, or an invasive (iEEG) setup, and estimates synchronization R. The deviation of current parameters from the optimal value (R˜0.5) of synchronization is calculated. The deviation from this value correlates with a change in excitability; higher values indicate an increased excitability, lower values indicate a decrease in excitability relative to normal values. A tolerance range will be introduced for tolerable degrees of deviation (e.g. 10%). Feedback signals to the human, which will often be a medical worker testing a patient, about the absolute synchronization value R and the deviation of the current brain state from the normal value (R˜0.5) will be provided. In certain embodiments, recorded EEG will be evaluated during clinical visits, and if the synchronization value has changed from the some predetermined, patient-individual value, an alert will be issued signaling, for example, that the subject's excitability has changed which can results in an increased risk of epileptic seizures. The objective biomarker for excitability provided by the system claimed in this patent, will inform the medical worker or other person about the current state of his/her patients excitability. By providing this objective marker with a reference point for normal conditions, therapy can be adapted, monitored and controlled. This system proposed in this patent will allow individualized medicine treatment.

In a first aspect, the method features methods of continuously monitoring neuronal synchronization in a subject comprising (a) determining a deviation in synchronization R from a predetermined value at rest, wherein the pre-determined value of R is 0.5; and (b) repeating step (a) one or more times to continuously monitor neuronal avalanches in a subject.

In one embodiment, the method further comprises (c) identifying the variability H of the measured synchronization R over time. In another embodiment, step (a) comprises (i) continuously recording the electroencephalogram (EEG); (ii) filtering the EEG; (iii) calculating the instantaneous synchronization as a function of time across different channels in this frequency band; (iv) calculating the mean synchronization R as the average of the instantaneous synchronization over time; (v) calculating the variability of synchronization H.

In another embodiment, the method features methods to compare measurements and values of synchronization R and variability of synchronization H over multiple recording sessions that can be several hours, several day, several weeks or years apart from each other. The method provides a record of these R and H values at all times when EEG was recorded and allows to display a history of all values in the past.

In another embodiment, the EEG is continuously recorded at more than one site, for example 2, 3, 4, 5, 6, 7, 8, 9, 10, 11, 12, 13, 14, 15, 16, 17, 18, 19, 20, 21, 22, 23, 24, 25 or more sites. In a related embodiment, the EEG is continuously recorded at more than 10 sites.

In a further embodiment, the EEG is filtered between 50-100 Hz.

In a further embodiment, the EEG is filtered between 1-50 Hz, or 1-100 Hz, or 1-4 Hz, or 4-8 Hz, or 8-12 Hz, or 12-25 Hz, or 25-50 Hz, or 100-200 Hz, or 200-400 Hz, or any other frequency band.

In one embodiment, it can be tested if and what antiepileptic drugs work and to what quantitative extent the work in an individual patient. The synchronization value R related to the brain's excitability will provide a directly accessible biomarker.

In another embodiment, the invention features the method to determine whether antiepileptic drugs have been taken in a regular, prescribed manner reflected by the expected levels of excitability quantified by synchronization R.

In yet another embodiment, the invention is used to identify patients that do not respond to a certain antiepileptic drug or, possibly, any antiepileptic drugs due to the failure to induce a decrease in excitability, i.e. synchronization R.

In another embodiment, the invention is used by a medical doctor/assistant/nurse to evaluate the effectiveness of medical treatment with antiepileptic drugs and to provide a record of disease progress and AED function of time and over multiple recording sessions. The synchronization R provides feedback as to the patient's excitability level over long times and multiple recording sessions.

In another embodiment, the variability of synchronization H is used as a biomarker for the cognitive deficits either induced by AED or other drugs, or related to psychiatric diseases.

In another aspect, the invention features a method of determining the degree of sleep deprivation in a subject comprising (a) determining the increase in synchronization (R) from a predetermined value at rest, wherein the pre-determined value of R is the synchronization R and the predetermined value is 0.5; and (b) repeating step (a) one or more times, wherein a change in R from the pre-determined value indicates the degree of sleep deprivation in a subject.

In yet another aspect, the invention features a method of identifying subjects that are susceptible to a sleep disorder comprising (a) determining a deviation in synchronization (R) from a predetermined value at rest, wherein the pre-determined value of R is the synchronization R and the predetermined value is 0.5; and (b) repeating step (a) one or more times, wherein a change in R from the pre-determined value indicates that the subject is susceptible to a sleep disorder.

In one embodiment, the subject is suffering from a sleep disorder.

In another embodiment of the above aspects, the method further comprises gathering data from other physiological sensors.

In a further embodiment of the above aspects, the method is operational with hardware or software or a combination thereof.

### DEFINITIONS

To facilitate an understanding of the present invention, a number of terms and phrases are defined below.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “behavioral performance” is meant to refer to performance in a cognitive task, such as, but not limited to, reaction time in a typical psychomotor vigilance task (PVT), a sensorimotor coordination task, such as steering a vehicle through demanding environment, or cognitive functions, such as decision making

The term “continuously monitoring” is meant to refer to determining a value or output more than one time, for example two, three, four, five, six, seven, eight, nine, ten or more times with relatively short intervals between consecutive measurements.

The term “electroencephalogram (EEG)” is meant to refer to the recording of electrical activity, typically along the scalp, but also measured subdurally.

The term “sleep disorder” is meant to refer to generally any abnormal sleeping pattern. Examples of sleep disorders include, but are not limited to, dyssomnia, insomnia, sleep apnea, narcolepsy, and circadian rhythmic disorders.

The term “subject” is meant to refer to any form of animal. Preferably the subject(s) are mammal, and most preferably human.

The term “synchronization” or “mean synchronization” refers to phase synchronization across different brain regions. This includes synchronization measures such as the Kuramoto order parameter, the mean Kuramoto order parameter, 6mean phase coherence, phase coherence values, cross correlation, mean cross correlation, phase-locking based measures, phase-locking intervals, pearson correlation, lag-correlation.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1. Intrinsic measures of synchronization correlate with the size of stimulation evoked responses. a, Evoked responses to subdural electrical stimulation were used to directly infer cortical excitability. The plot shows a representative example of the mean evoked response from one electrode of patient 1. Excitability is reflected by the size of the evoked potential and was quantified as the absolute difference A between positive and negative maxima of the evoked response. Electrical stimulation was continuously applied (approx. 3 Hz) for multiple hours allowing continuous measurement of cortical excitability. Unperturbed segments before the stimulation (grey bar) were used to calculate synchronization R across different electrodes. b, Time course of stimulation evoked response A and synchronization R of ongoing cortical activity over multiple hours in patient 1. c, Evoked response amplitude A and synchronization R are highly correlated across broad frequency bands. Black line shows linear regression, R² reflects the goodness of fit. d, Summary of the quality of fit for different frequency ranges of patient 1 and patient 2. FIG. 2 shows the power law exponent is close to α=−3/2 at the critical branching parameter σ=1. Phase plot of the power law exponent, a, versus the branching parameter, a. Each point represents the mean across datasets and error bars represent SEM.

FIG. 2. Intrinsic measures of synchronization track antiepileptic drug (AED) action during multi-day recordings. a1-a4, Markers and level of medication of four patients. Top: AED dosage. Below: changes in mean phase synchronization (R) for the frequency band 50-100 Hz. Round markers correspond to one hour measurements (red markers signify that at least one epileptic seizure occurred during this one hour, grey markers no seizures). Daily averages were taken over the 12 highest hours of each day and are plotted as bars. Light colors were used when recordings did not encompass a full 24 hour day. Error bars on each solid bar indicate standard error of the mean. Time on the x-axis is labeled in days where each day starts at midnight. b, Differences between full days of low and high AED levels for all 10 patients. *p≦0.05$, ** p≦0.001, two-sided paired sample t-test.

FIG. 3. Antiepileptic drugs (AED) shift network dynamics from moderate synchrony and peak variability to states with low synchrony and limited variability. a, Combined data from 10 patients and different frequency bands. Left vertical axis: Variability (H) as a function of mean synchronization (R) (grey round markers). H peaks at moderate R. Right vertical axis: Histogram of time (hours) spent at different levels of mean synchronization. Without AED (black histogram bars), network dynamics predominantly settles at moderate synchronization levels with peak variability. With AED (blue histogram bars), network dynamics spends more time in low synchrony states with decreased variability. b, Averages of mean (R, left plot) and variability (H, right plot) of synchronization for hours without (left) and with (right) AED. *p≦0.05$, ** p≦0.001, two-sided independent sample t-test. c, Illustration of the behavior of measures R and H as a function of the ratio of excitation and inhibition (E/I) or, more generally, network excitability. As excitability is pharmacologically increased from disfascilitated to disinhibited dynamics, R increases while H peaks at a normal, physiological E/I ratio. This qualitative behavior was observed in cortical cultures in vitro and in rodents in vivo and is in line with observations under different levels of AED reported here. The combined behavior of R and H in our data suggests that AED drive the network toward a more disfacilitated/inhibited state (blue arrow).

### DETAILED DESCRIPTION

The synchronization metrics can be derived from ongoing activity recorded from EEG or MEG or other neuronal activity measurement. The mean level of synchronization reflect normal brain activity in the awake state and is characterized by a value of around 0.5. In contrast to EEG power or other markers, the synchronization metric is an absolute metric. This allows the metric to be used in absolute terms, i.e. no control group is required to identify performance changes. The present invention demonstrates that mean synchronization R correlates strongly with a) brain excitability determined by stimulation experiments and b) with the number and dosage of antiepileptic drugs applied (see example in this patent).

Described herein is a clinical biomarker to monitor the brain's excitability. Excitability is often changed in health and disease, as such an objective marker is missing. In patients suffering from epilepsy, the reduction of brain excitability with antiepileptic drugs (AED) is the prime and first-line treatment option. As such, effective clinical treatment and diagnosis would benefit from objective biomarkers quantifying excitability and the effect of AED on excitability. However, prior to the invention described herein, there was a lack of objective biomarkers measuring excitability. The results described herein demonstrated that a marker relating cortical dynamics to excitability. As described herein, mean synchronization highly correlates with the level of excitability in the brain. In this regard, the metrics provide an objective marker for excitability and have the potential to guide and monitor the effect of treatment to improve the epilepsy condition in clinical settings. Software extracting the markers related to synchronization is a useful tool for diagnostic and monitoring treatment progress in clinical settings.

### Methods

### Mean Synchronization R

Synchronization between different EEG channels can be measured as phase-synchronization. In order to derive a phase-synchronization value, signals have to first be filtered in a frequency band. Estimates of mean of phase synchronization for band-pass filtered data are then derived for band-pass filtered data. After filtering the data in the respective frequency band, one first obtains a phase trace θi(t) from each EEG trace Fi(t) by applying its Hilbert transform H[Fi(t)]

$${θ}_{i} ( t ) = arctan \frac{H [ {F}_{i} ( t ) ]}{{F}_{i} ( t )} . \qquad [ 1 ]$$

Next, quantifies the mean synchrony R in each ECoG or EEG segment by

$$R = 〈 r ( t ) 〉 = \frac{1}{L} {\sum }_{t = 1}^{L} r ( t ) , \qquad [ 2 ]$$

where L is the length of the data segment in samples and r(t) is the Kuramoto order parameter

$$r ( t ) = \frac{1}{N} \vert {\sum }_{j = 1}^{N} {e}^{{iθ}_{j} ( t )} \vert \qquad [ 3 ]$$

which is used as a time-dependent measure of phase synchrony. Here, N is the number of ECoG or EEG channels in the data segment. The length of the segment in samples L is the product of the time segment considered and the sampling frequency.

It is noted that other means to derive phases or phase-synchronization are included in the patent claim. This includes wavelet-based synchronization measures, wavelet transform based measures.

It is noted that other measures of synchronization are also included in this patent. This includes synchronization measures such as mean phase coherence, phase coherence values, cross correlation, mean cross correlation, phase-locking based measures, phase-locking intervals, pearson correlation, lag-correlation.

### Variability of Synchronization H

As a measure for the variability of synchronization on derives the entropy of r(t) in each segment by

$$H ( r ( t ) ) = - {\sum }_{i = 1}^{B} {p}_{i} {log}_{2} {p}_{i} , \qquad [ 4 ]$$

where one estimates a probability distribution of r(t) by binning values into intervals. pᵢ is then the probability that r(t) falls into a range bᵢ<r(t)≦bᵢ+1. Similar to [18, 11], results are robust over a broad range for the number of bins B used. We applied B=24 bins in the current analysis. In the realm of this application, other bin numbers such as B=2, 3, 4, 5, 6, 7, 8, 9, 10 or any other number are also included.

In certain aspects, the present invention features methods of continuously or discontinuously monitoring mean synchronization and variability of synchronization in a subject. In preferred embodiments, the method comprises (a) determining a deviation in mean synchronization (R) from a predetermined value at rest, wherein the pre-determined value of R is 0.5, and the variability of synchronization H; and (b) repeating step (a) one or more times (e.g. 1, 2, 3, 4, 5, 6, 7, 8, 9, 10, 11, 12, 13, 14,15, 16, 17, 18, 19, 20, 25, 30, 35, 40, 45, 50 or more times) to continuously or discontinuously monitor synchronization R and its variability H in a subject.

EEG signals can be obtained by any method known in the art, or subsequently developed by those skilled in the art to detect these types of signals. Sensors include but are not limited to electrodes or magnetic sensors. Preferably, the EEG is continuously recorded at >10 sites.

The EEG recording is characterized by amplitude, frequency and their change over time. The frequency component of the EEG can be utilized to infer the level of an individual's neural activity. The frequencies are broken down into ranges which describe how alert and conscious a person is at any given time. The delta frequency (1-4 Hz) is associated with deep sleep. The theta frequency (4-5 Hz to 8-9 Hz) is associated with drowsiness, and delta activity is also common. The alpha frequency (8-13 Hz) is associated with relaxed wakefulness, where not much brain resources are devoted to any one thing. The beta frequency (12-20 Hz, or 30 Hz) and the gamma frequency (36-44 Hz) are associated with alert attentiveness

In certain embodiments, the EEG is filtered between 50-100 Hz.

If electrodes are used to pick up the brain wave signals, these electrodes may be placed at one or several locations on the subject(s)' scalp or body. The electrode(s) can be placed at various locations on the subject(s) scalp in order to detect EEG or brain wave signals. Common locations for the electrodes include frontal (F), parietal (P), anterior (A), central (C) and occipital (0). Preferably for the present invention at least one electrode is placed in the occipital position. In order to obtain a good EEG or brain wave signal it is desirable to have low impedances for the electrodes. Typical EEG electrodes connections may have an impedance in the range of 5 to 10 K ohms. It is in general desirable to reduce such impedance levels to below 2 K ohms. Therefore a conductive paste or gel may be applied to the electrode to create a connection with an impedance below 2 K ohms. Alternatively, the subject(s) skin may be mechanically abraded, the electrode may be amplified or a dry electrode may be used. Dry physiological recording electrodes of the type described in U.S. patent application Ser. No. 09/949,055 are herein incorporated by reference. Dry electrodes provide the advantage that there is no gel to dry out, no skin to abrade or clean, and that the electrode can be applied in hairy areas such as the scalp. Additionally if electrodes are used as the sensor(s), preferably at least two electrodes are used--one signal electrode and one reference electrode; and if further EEG or brain wave signal channels are desired the number of electrodes required will depend on whether separate reference electrodes or a single reference electrode is used. For the various embodiments of the present invention, preferably an electrode is used and the placement of at least one of the electrodes is at or near the occipital lobe of the subject's scalp.

In one embodiment, the method further comprises (c) identifying the variability H of the measured synchronization R over time. In another embodiment, step (a) comprises (i) continuously recording the electroencephalogram (EEG); (ii) filtering the EEG; (iii) calculating the instantaneous synchronization as a function of time across different channels in this frequency band; (iv) calculating the mean synchronization R as the average of the instantaneous synchronization over time; (v) calculating the variability of synchronization H.

In another embodiment, the method features methods to compare measurements and values of synchronization R and variability of synchronization H over multiple recording sessions that can be several hours, several day, several weeks or years apart from each other. The method provides a record of these R and H values at all times when EEG was recorded and allows to display a history of all values in the past.

In another embodiment, the EEG is continuously recorded at more than one site, for example 2, 3, 4, 5, 6, 7, 8, 9, 10, 11, 12, 13, 14, 15, 16, 17, 18, 19, 20, 21, 22, 23, 24, 25 or more sites. In a related embodiment, the EEG is continuously recorded at more than 10 sites.

In a further embodiment, the EEG is filtered between 50-100 Hz.

In a further embodiment, the EEG is filtered between 1-50 Hz, or 1-100 Hz, or 1-4 Hz, or 4-8 Hz, or 8-12 Hz, or 12-25 Hz, or 25-50 Hz, or 100-200 Hz, or 200-400 Hz, or any other frequency band.

In one embodiment, it can be tested if and what antiepileptic drugs work and to what quantitative extent the work in an individual patient. The synchronization value R related to the brain's excitability will provide a directly accessible biomarker.

In another embodiment, the invention features the method to determine whether antiepileptic drugs have been taken in a regular, prescribed manner reflected by the expected levels of excitability quantified by synchronization R.

In yet another embodiment, the invention is used to identify patients that do not respond to a certain antiepileptic drug or, possibly, any antiepileptic drugs due to the failure to induce a decrease in excitability, i.e. synchronization R.

In another embodiment, the invention is used by a medical doctor/assistant/nurse to evaluate the effectiveness of medical treatment with antiepileptic drugs and to provide a record of disease progress and AED function of time and over multiple recording sessions. The synchronization R provides feedback as to the patient's excitability level over long times and multiple recording sessions.

In another embodiment, the variability of synchronization H is used as a biomarker for the cognitive deficits either induced by AED or other drugs, or related to psychiatric diseases.

In another aspect, the invention features a method of determining the degree of sleep deprivation in a subject comprising (a) determining the increase in synchronization (R) from a predetermined value at rest, wherein the pre-determined value of R is the synchronization R and the predetermined value is 0.5; and (b) repeating step (a) one or more times, wherein a change in R from the pre-determined value indicates the degree of sleep deprivation in a subject.

In yet another aspect, the invention features a method of identifying subjects that are susceptible to a sleep disorder comprising (a) determining a deviation in synchronization (R) from a predetermined value at rest, wherein the pre-determined value of R is the synchronization R and the predetermined value is 0.5; and (b) repeating step (a) one or more times, wherein a change in R from the pre-determined value indicates that the subject is susceptible to a sleep disorder.

In one embodiment, the subject is suffering from a sleep disorder.

In another embodiment of the above aspects, the method further comprises gathering data from other physiological sensors.

In a further embodiment of the above aspects, the method is operational with hardware or software or a combination thereof.

Preferably, the subject(s) are mammal, and more preferably human. The methods described herein can be used in subjects that experience prolonged periods of wakefulness (e.g. the subject has not slept for 24, 36, 48, 72 or more hours), for example, but not limited to, subjects on duty and in patients with sleep disorders. Typical applications may be related to many civil and military professions. Other subjects may be those post-exercise, wherein the methods described herein are used to identify individuals resilient to sleep deprivation or at risk.

The subjects of the present invention may be suffering from a sleep disorder. A sleep disorder is meant to refer to any abnormal sleeping pattern. Examples of sleep disorders include, but are not limited to, dyssomnia, insomnia, sleep apnea, narcolepsy, and circadian rhythmic disorders.

In any of the methods described herein, the method may comprise a further step of gathering data from other physiological sensors of brain activity. For example magnetoencephalography (MEG), functional MRI (fMRI) using the BOLD signal or other related measures, optical imaging using fluorescent dyes that track neuronal activity such as intracellular calcium sensors, implanted microelectrode arrays to record the local field potential (LFP) or electrocorticogram (ECoG). In other embodiments, the method may comprise a step of gathering data related to typical signs of sleepiness, such as increased eye blink and/or yawning frequency.

### On-Line Evaluation

The methods of the present invention can be operational with numerous other general purpose or special purpose computing system environments or configurations. Examples of well known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, smart phones, network PCs, minicomputers, mainframe computers, distributed computing environments that comprise any of the above systems or devices, and the like.

The methods of the present invention can be described in the general context of computer instructions, such as program modules, being executed by a computer. Generally, program modules comprise routines, programs, objects, components, data structures, etc. that perform particular tasks or implement particular abstract data types. The systems and methods of the present invention can also be practiced in distributed computing environments where tasks are performed by remote processing devices that are linked through a communications network. In a distributed computing environment, program modules can be located in both local and remote computer storage media including memory storage devices.

The methods of the present invention can be operational with hardware or software to allow continuous monitoring of subjects, for example subjects under extended wake periods, or discontinuous monitoring, for example patients that visit their doctor every couple of days, weeks, months, or years. The proposed metrics will be implemented in software (IEMS=intrinsic excitability measures software) or hardware and will allow continuous or discontinuous monitoring of subjects. One skilled in the art will appreciate that the methods disclosed herein can be implemented via a general-purpose computing device in the form of a computer. The components of the computer can comprise, but are not limited to, one or more processors or processing units, a system memory, and a system bus that couples various system components including the processor to the system memory. Further, the methods of the present invention can be operational with numerous general purpose or special purpose computing system environments or configurations. Examples of well-known computing systems, environments, and/or configurations that can be suitable for use with the systems and methods comprise, but are not limited to, personal computers, server computers, laptop devices, smartphones, and multiprocessor systems. Additional examples comprise set top boxes, programmable consumer electronics, network PCs, minicomputers, mainframe computers, distributed computing environments that comprise any of the above systems or devices, and the like.

The methods of the present invention can be described in the general context of computer instructions, such as program modules, being executed by a computer. Generally, program modules comprise routines, programs, objects, components, data structures, etc. that perform particular tasks or implement particular abstract data types. The systems and methods of the present invention can also be practiced in distributed computing environments where tasks are performed by remote processing devices that are linked through a communications network. In a distributed computing environment, program modules can be located in both local and remote computer storage media including memory storage devices.

One skilled in the art will appreciate that the systems and methods disclosed herein can be implemented via a general-purpose computing device in the form of a computer. The components of the computer can comprise, but are not limited to, one or more processors or processing units, a system memory, and a system bus that couples various system components including the processor to the system memory.

The system bus represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (USA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus, and all buses specified in this description can also be implemented over a wired or wireless network connection and each of the subsystems, including the processor, a mass storage device, an operating system, IEMS software, neuronal data, a network adapter, system memory, an Input/Output Interface, a display adapter, a display device, and a human machine interface, can be contained within one or more remote computing devices at physically separate locations, connected through buses of this form, in effect implementing a fully distributed system.

The computer typically comprises a variety of computer readable media. Exemplary readable media can be any available media that is accessible by the computer and comprises, for example and not meant to be limiting, both volatile and non-volatile media, removable and non-removable media. The system memory comprises computer readable media in the form of volatile memory, such as random access memory (RAM), and/or non-volatile memory, such as read only memory (ROM). The system memory typically contains data such as neuronal data and/or program modules such as operating system and IEMS software that are immediately accessible to and/or are presently operated on by the processing unit.

The computer can also comprise other removable/non-removable, volatile/non-volatile computer storage media. For example, and not meant to be limiting, a mass storage device can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device, including by way of example, an operating system and IEMS software. Each of the operating system and IEMS software (or some combination thereof) can comprise elements of the programming and the IEMS software. Neuronal data can also be stored on the mass storage device. Neuronal data can be stored in any of one or more databases known in the art. Examples of such databases comprise, DB2, MICROSOFT Access, MICROSOFT SQL Server, ORACLE, mySQL, PostgreSQL, and the like. The databases can be centralized or distributed across multiple systems.

The user can enter commands and information into the computer via an input device (not shown). Examples of such input devices comprise, but are not limited to, a keyboard, pointing device (e.g., a “mouse”), a microphone, a joystick, a scanner, and the like. These and other input devices can be connected to the processing unit via a human machine interface that is coupled to the system bus, but can be, connected by other interface and bus structures, such as a parallel port, game port, an IEEE 1394. Port (also known as a Firewire port), a serial port, or a universal serial bus (USB).

A display device can also be connected to the system bus via an interface, such as a display adapter. It is contemplated that the computer can have more than one display adapter and the computer can have more than one display device. For example, a display device can be a monitor, an LCD (Liquid Crystal Display), or a projector. In addition to the display device, other output peripheral devices can comprise components such as speakers (not shown) and a printer (not shown) which can be connected to the computer via Input/Output Interface.

A neuronal activity detector can communicate with the computer via Input/Output Interface or across a local or remote network. In one aspect, users utilize a neuronal activity detector that is capable of collecting neuronal data. It will be appreciated that the neuronal activity detector can be any type of neuronal activity detector, for example and not meant to be limiting, a microelectrode array (to record LFPs and single/multi-unit activity), a surface electrode system (to record the EEG or ECoG), a charge-coupled device camera (CCD) or photodiode array (to record activity-dependent fluorescence changes), a magnetometer type SQUID (superconducting quantum interference device) sensor (to record the MEG), a functional magnetic resonance imaging (fMRI) device to measure the activity related blood oxygen-level dependent signal (BOLD), and the like. In another aspect, the neuronal activity detector can be an independent stand alone device, or can be integrated into another device. Optionally, the communication with computer via Input/Output Interface can be via a wired or wireless connection.

The computer can operate in, a networked environment using logical connections to one or more remote computing devices. By way of example, a remote computing device can be a personal computer, portable computer, a server, a router, a network computer, a peer device or other common network node, and so on. Logical connections between the computer and a remote computing device can be made via a local area network (LAN) and a general wide area network (WAN). Such network connections can be through a network adapter. A network adapter can be implemented in both wired and wireless environments. Such networking environments are conventional and commonplace in offices, enterprise-wide computer networks, intranets, and the Internet.

An implementation of IEMS software can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any method or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

The processing of the disclosed systems and methods of the present invention can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

The IEMS Software allows for the study of synchronization measures and includes many analysis features. IEMS Software allows for the calculation of mean synchronization (R), the time course of synchronization (r(t)) and the variability of synchronization (H). A multi-function control window contains functions that extract the synchronization parameters R and H. The history of values R and H from past recordings can also be displayed. The reference point R=0.5 will be marked. Additional features relate to the identification and labeling of recording locations to superficial cortical layers in which synchronization is recorded. IEMS Software allows for the storage of spatial information, e.g. images, and miscellaneous data specific to an experimental configuration. IEMS Software allows to be applied only to certain EEG channels chosen by the user or all channels.

IEMS Software can analyze the current synchronization from different regions in the brain defined by different sets of channels. IEMS Software allows the used to set different filter bands for the calculation of R, H and r(t). It can display results depending on the different filter setting.

Apparatus

According to various embodiments, an EEG headset is provided to subjects for use at home, recreational, at work, as well as in laboratory environments. In particular embodiments, the EEG headset includes multiple dry electrodes individually isolated and amplified. Data from individual electrodes may be processed prior to continuous transmission to a data analyzer. The continuously recorded EEG can be evaluated online as described herein and in US 20090036791, incorporated by reference in its entirety herein. For example, the methods described herein are used by consumers to monitor excitability in real time, e.g., on a smart phone.

Typical applications of the methods described herein are related to many civil and military professions, although not limited as such. A subject may wear the portable neuro-response data collection mechanism during a variety of activities in non-laboratory settings. This allows collection of data from a variety of sources while a subject is in a natural state. For example, dry EEG electrodes can be easily integrated into helmets of pilots and soldiers to monitor the EEG. The present inventors have demonstrated that the avalanche metric is evident even when using a relatively small set of sensors.

In certain aspects, EEGs are recorded by a wireless EEG headset.

In certain aspects, the invention features an integrated program that includes the methods described herein performed with an EEG headset, for example a wireless headset. The methods can be performed in the comfort of the subject's home or workplace. The data are reviewed by a specialist after upload and can be used for diagnosis or intervention, and can be made through an integrated web-portal. The portal allows for on-going clinician monitoring of progress.

### EXAMPLES

It should be appreciated that the invention should not be construed to be limited to the example that is now described; rather, the invention should be construed to include any and all applications provided herein and all equivalent variations within the skill of the ordinary artisan.

### Measuring Brain Excitability in Epilepsy Patients

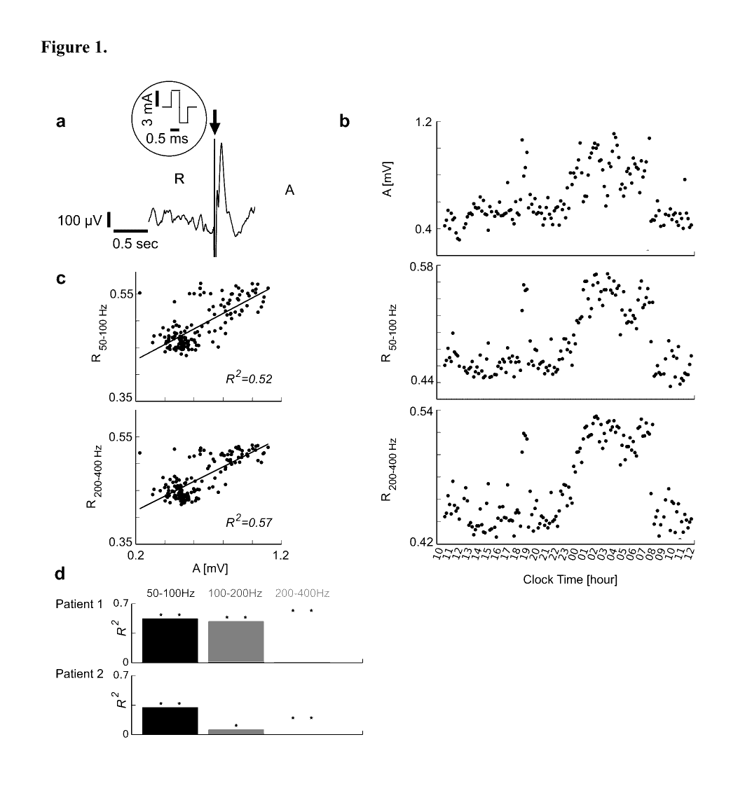

To evaluate the correlation between synchronization R measured from ongoing activity and more direct, current state-of-the-art measures of brain excitability we probed cortical excitability in human electrocorticogram in the most direct way by electrical stimulation. Previous work has shown that the amplitude of evoked cortical potentials by short pulses of electrical stimulation is a direct measure of cortical excitability: while small amplitudes indicate a comparably small excitability, large responses suggest excitability to be high [3, 2, 22]. We designed a stimulation protocol which allowed us to measure electrical stimulation evoked responses as a direct marker of excitability, as well as phase synchronization of ongoing activity as a potential intrinsic excitability measure over long periods of time within individual patients. FIG. 1a shows a typical evoked response in one channel. The amplitude A of evoked potentials, measured from highest peak to lowest trough, exhibited considerable variation (FIG. 1b) indicating varying levels of excitability over the course of hours. Unperturbed time segments before each stimulus (FIG. 1a, grey bar) were used to calculate phase synchronization in different frequency bands. We observed that mean synchro-nization levels R followed a very similar time course (FIG. 1b) which was reflected in high correlation values between amplitude A and synchronization R (FIG. 1c). This significant correlation was observed across a broad range of frequencies from 50 to 400 Hz and in n=2 patients under investigation (FIG. 1d). Throughout the manuscript, we will focus on IEM in this frequency range, i.e. the bands 50-100 Hz, 100-200 Hz and 200-400 Hz. These results indicate that mean levels of phase synchronization are directly related to cortical ex-citability in humans and consequently suggest them as valid indicators of excitability based on ongoing cortical activity.

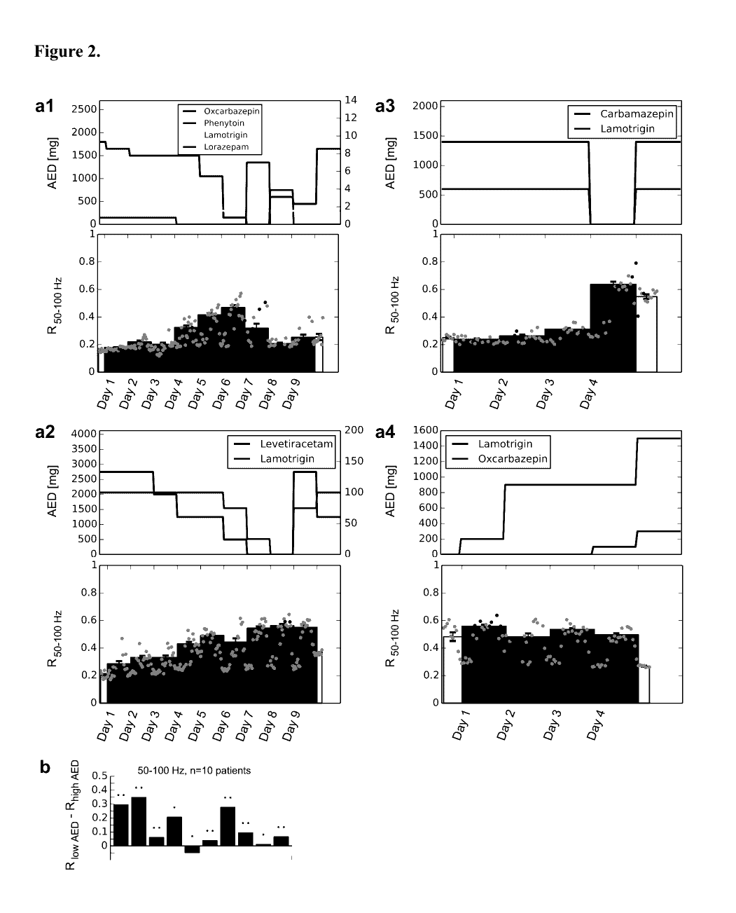

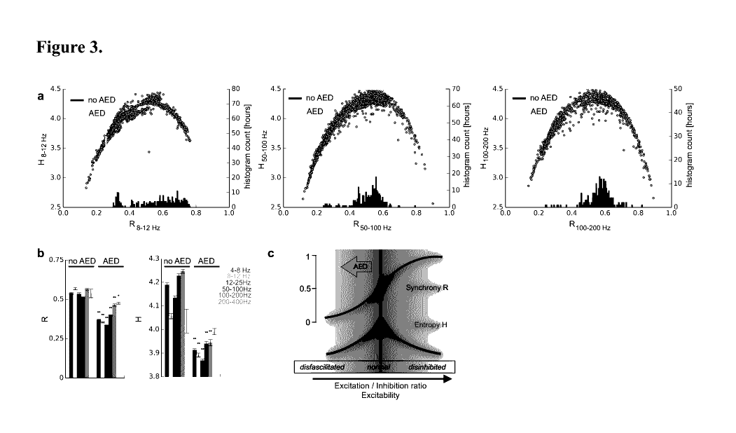

Next, we analyzed invasive EEG from ten patients undergoing presurgical monitoring during which antiepileptic medication had been varied. No stimulation was performed in these patients. The type of antiepileptic drugs used during this time, their dosages, and the time course by which they were tapered off were solely determined by clinical considerations and varied between patients. We were interested whether synchronization measures would, analogously to in vitro analyses under pharmacological manipulation, exhibit an AED-dependent trajectory which would be consistent with the hypothesis of a change in E/I balance. We thereby focused particularly on mean levels of synchronization R in the frequency range of 50-100 Hz since stimulation analysis had revealed very good correlations with evoked responses in this frequency band and because this frequency range could also be resolved in datasets recorded with lower sampling rates. In the following we will therefore use R50-100Hz as the primary intrinsic excitability measure although results were generally robust over a broader range of frequencies (FIG. 3). Mean synchronization R exhibited considerable variability during the multi-day recordings in each patient. FIG. 2a shows time courses for four representative patients. Typically, R was low during days with high AED load and increased when AEDs were reduced. The time course of R thereby closely followed an inverse relation with antiepileptic drug load. Since especially the highest R values showed a strong dependence on AED, we averaged over each day's highest 12 hours to determine a daily mean (FIG. 2a, solid bars). To quantify the visually observed dependence of R on AED dosage (FIG. 2a), we compared R values from one day of highest AED load (high AED) to the day with the lowest dose of AED (low AED) in each patient. The day with high AED load was usually the first full day of recording. When there was more than one day with the same amount of low or none AED, we chose the one furthest away from the high AED day. Statistical analysis revealed a significant in-crease in R from high to low levels of AED for the majority of patients (FIG. 2b, two-sided paired sample t-test).

Previous in vitro studies had suggested that normal cortical dynamics under a physiological E/I balance is, besides moderate levels of the mean, characterized by a maximum in variability of synchronization [18]. Here, we quantified variability of synchronization by its entropy H and observed that peak variability was typically found at moderate mean levels (FIG. 3a, grey circles). For R values much smaller or larger than R=0.5 variability dropped to smaller values for all fre-quency bands investigated. To gain better insight into the function of AEDs on network dynamics, we separated days on which no AED had been given from days where AED had been applied. Notably, we found that cortical network activ-ity without AED typically settled at these moderate R levels where R≅0.5 and variability peaks (FIG. 3a, black bars in histogram). Conversely, during times when AED had been ad-ministered, we observed that markedly more time was spent at lower R values with decreased variability as is evident by the left-shift visible in the histogram (FIG. 3a, blue bars in his-togram). Comparison between “no AED” and “AED” hours revealed a significant decrease from R≅0.5 to lower values across a broad range of frequencies along with a drop in vari-ability H (FIG. 3b, two-sided independent sample t-test). FIG. 3c schematically summarizes the qualitative behavior of in-trinsic excitability measures R and H as a function of the ratio of excitation and inhibition (E/I) or, more generally, network excitability as observed in our data in human invasive EEG recordings as well as in cortical cultures in vitro and in rodents in vivo [19, 20, 23, 18]. As excitability is pharmacologically increased from disfascilitated to disinhibited dynamics, R increases while H peaks at a normal, physiological E/I ratio. In our data we observed an increase of R and H when antiepilep-tic drug load was reduced. This is in line with observations in cortex cultures and provides strong indication for an increase in excitability in cortical networks when AEDs are tapered off. These analogies suggest that excitability is effectively reduced by AED (FIG. 3c, blue arrow) and contributes to the strong correlation with stimulation evoked responses reported above which provides further support for R as a reliable measure of cortical excitability.

### Materials and Methods

The experiments described herein were carried out with, but are not limited to, the following materials and methods.

The stimulation dataset (1) was used to directly measure cortical excitability based on activity responses following electrical stimulation and investigate its relation to intrinsic excitability measures. The long duration over which electrical stimulation was applied in these recordings allowed to correlate the response to stimulation, which is often taken as a direct measurement of cortical excitability, to the synchronization measures derived from ongoing activity. Data were collected from two patients suffer-ing from focal epilepsies undergoing evaluation for the surgical resection of epileptic foci at St. Vincent's Hospital in Melbourne, Australia. Ethics approval was obtained from St. Vincent's Human Research Ethics Committee. Intracranial electrodes (N=24 electrodes, patient 1) as well as subdural grid and strip electrodes (N=93 electrodes, patient 2) were used. Data were sampled at 5000 Hz. The stimulation protocol has been described previously [28] and was followed here closely. The stimulation protocol consisted of blocks of stimulations with biphasic electrical pulses of 3 mA amplitude and 0.5 ms pulse width which were delivered to two electrodes in each patient with a stimulation frequency of 0.3 Hz (every 15050 sampling points) and referenced against a third electrode. Each stimulation block consisted of 100 stimulations and was re-peated every 10 minutes over at least 24 hours in each patient over which data was continuously recorded for offline analysis. For further offline analysis, the two stimu-lation electrodes, the reference electrode as well as other electrodes showing signs of large stimulation artifacts or epileptic discharges were excluded. Stimulation-evoked potentials in each channel were derived by averaging the responses in each stimulation block (100 stimulations) time-locked to the onset of stimulation and applying a band pass filter (third order butterworth filter; 0.01-100 Hz in patient 1, 1-100 Hz in patient 2 due to sometimes appearing slow current transients) in the reverse time direction so that potential stimulation artifacts or ringing would end up before the stimulation. In each patient, two channels showing strong stimulation evoked responses were chosen and their mean amplitude A, defined as the distance from peak to trough in each channel (FIG. 1a), was taken as a measure of excitability [3]. Mean synchronization, R, was calculated across channels from 950 ms long segments preceding and leaving a 25 ms distance to the stimulation onset (−975 to −25 ms from stimulation onset). These segments were first filtered in the frequency band of choice using a phase neu-tral filter by applying a second order butterworth filter in both directions. A notch filter to eliminate line noise was applied subsequently. In patient 2, some stimulation blocks had to be removed from the analysis due to high frequency noise levels (approx. 1000-2000 Hz) occurring predominantly at the beginning of the stimulation protocol. Antiepileptic drugs were kept constant during the stimulation period.

The second electrocorticogram dataset (2) consisted of multi-day recordings from 10 patients undergoing presurgical monitoring at the Epilepsy Center of the University Hospital of Freiburg, Germany [40]. No stimulation was performed in these patients. All patients suffered from focal epilepsies. The number of electrodes varied between patients (from N=30 to N=114), included both surface and intracranial electrodes and their placement was solely determined by clinical considerations. The invasive EEG data were sampled at either 256 (n=4 patients), 512 (n=1 patient) or 1024 Hz (n=5 patients). To capture dynamic changes in our markers, EEG data were analyzed in segments of 10 minutes duration. To prevent aliasing and to eliminate possible line noise and low frequency components, the EEG data were preprocessed by a 50 Hz notch filter and a phase-neutral band pass filter in the frequency band of choice (phase neutral filter by applying a second order butterworth filter in both directions). A small number of 10 min segments were excluded from the analysis due to artifacts presenting as high noise levels in the approx. 25-30 Hz frequency range across electrodes.

We derived estimates of mean and variability of phase synchronization for band-pass filtered data in all three datasets. After filter-ing the data in the respective frequency band, we first obtained a phase trace θi(t) from each ECoG or EEG trace Fi(t) by applying its Hilbert transform H[Fi(t)]

$${θ}_{i} ( t ) = arctan \frac{H [ {F}_{i} ( t ) ]}{{F}_{i} ( t )} . \qquad [ 1 ]$$

Next, we quantified the mean synchrony R in each ECoG or EEG segment by

$$R = 〈 r ( t ) 〉 = \frac{1}{L} {\sum }_{t = 1}^{L} r ( t ) , \qquad [ 2 ]$$

where L is the length of the data segment in samples and r(t) is the Kuramoto order parameter

$$r ( t ) = \frac{1}{N} \vert {\sum }_{j = 1}^{N} {e}^{{iθ}_{j} ( t )} \vert \qquad [ 3 ]$$

which was used as a time-dependent measure of phase synchrony. Here, N is the number of ECoG or EEG channels in the data segment (see above). The length of the segment in samples L is the product of the time segment considered and the sampling frequency. As such it ranged from 4750 samples for the 950 ms segments used in dataset 1 to the 10 minute intervals multiplied by the respective sampling fre-quencies in dataset 2. When checking results for different interval lengths in dataset 2, we observed results to be robust for different interval lengths.

As a measure for the variability of synchronization we derived the entropy of r(t) in each segment by

$$H ( r ( t ) ) = - {\sum }_{i = 1}^{B} {p}_{i} {log}_{2} {p}_{i} , \qquad [ 4 ]$$

where we estimated a probability distribution of r(t) by binning values into in-tervals. pᵢ is then the probability that r(t) falls into a range bᵢ<r(t)≦bᵢ+1. Similar to [18, 11], we found results to be robust over a broad range for the number of bins B used. We applied B=24 bins in the current analysis.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

### REFERENCES

As should be understood, the citations above to one or more numbers within brackets (e.g., [1-3]) refers to the document(s) listed below first identified with a like number (thus, [1-3] above refers to the below listed Fenn et al.; Stickgold et al., and Walker et al. documents).
1. Valentin A, et al. (2002) Responses to single pulse electrical stimulation identify epileptogenesis in the human brain in vivo. Brain 125:1709-1718.
2. Valentin A, et al. (2005) Single-pulse electrical stimulation identifies epileptogenic frontal cortex in the human brain. Neurology 65:426-435.
3. Matsumoto R, et al. (2005) In vivo epileptogenicity of focal cortical dysplasia: a direct cortical paired stimulation study. Epilepsia 46:1744-1749.
4. Cantello R, et al. (2000) Cortical excitability in cryptogenic localization-related epilepsy: interictal transcranial magnetic stimulation studies. Epilepsia 41:694-704.
5. Werhahn K J, Lieber J, Classen J, Noachtar S (2000) Motor cortex excitability in patients with focal epilepsy. Epilepsy Res. 41:179-189.
6. Hamer H M, et al. (2005) Motor cortex excitability in focal epilepsies not including the primary motor arca-a TMS study. Brain 128:811-818.
7. Badawy R A, Vogrin S J, Lai A, Cook M J (2013) The cortical excitability profile of temporal lobe epilepsy. Epilepsia 54:1942-1949.
8. Bialer M, White H S (2010) Key factors in the discovery and development of new antiepileptic drugs. Nat Rev Drug Discov 9:68-82.
9. Badawy R A, Curatolo J M, Newton M, Berkovic S F, Macdonell R A (2006) Sleep deprivation increases cortical excitability in epilepsy: syndrome-specific effects. Neurology 67:1018-1022.
10. Huber R, et al. (2012) Human cortical excitability increases with time awake. Cereb Cortex.
11. Meisel C, Olbrich E, Shriki O, Achermann P (2013) Fading signatures of critical brain dynamics during sustained wakefulness in humans. J. Neurosci. 33:17363-17372.
12. Tononi G, Cirelli C (2014) Sleep and the price of plasticity: from synaptic and cellular homeostasis to memory consolidation and integration. Neuron 81:12-34.
13. Rothwell J C (1997) Techniques and mechanisms of action of transcranial stimulation of the human motor cortex. J. Neurosci. Methods 74:113-122.
14. Rossini P M, Rossi S (2007) Transcranial magnetic stimulation: diagnostic, therapeutic, and research potential. Neurology 68:484-488.
15. Rossi S, et al. (2009) Safety, ethical considerations, and application guidelines for the use of transcranial magnetic stimulation in clinical practice and research. Clin Neurophysiol 120:2008-2039.
16. Meisel C, Gross T (2009) Adaptive self-organization in a realistic neural network model. Phys Rev E 80:061917.
17. Meisel C, Storch A, Hallmeyer-Elgner S, Bullmore E, Gross T (2012) Failure of adaptive self-organized criticality during epileptic seizure attacks. PLoS Comput Biol 8:e1002312.
18. Yang H, Shew W L, Roy R, Plenz D (2012) Maximal variability of phase synchrony in cortical networks with neuronal avalanches. J Neurosci 32:10611072.
19. Castro-Alamancos M A (2000) Origin of synchronized oscillations induced by neocortical disinhibition in vivo. J. Neurosci. 20:9195-9206.
20. Castro-Alamancos M A, Rigas P (2002) Synchronized oscillations caused by disinhibition in rodent neocortex are generated by recurrent synaptic activity mediated by AMPA receptors. J. Physiol. (Lond.) 542:567-581.
21. Kitzbichler M G, Smith M L, Christensen S R, Bullmore E (2009) Broadband criticality of human brain network synchronization. PLoS Comput Biol 5:e1000314.
22. Enatsu R, et al. (2012) Cortical excitability varies upon ictal onset patterns in neocortical epilepsy: a cortico-cortical evoked potential study. Clin Neurophysiol 123:252-260.
23. Shew W L, Yang H, Petermann T, Roy R, Plenz D (2009) Neuronal avalanches imply maximum dynamic range in cortical networks at criticality. J Neurosci 9:15595-15600.
24. Finelli L A, Bauman H, Borb'ely A A, Achermann P (2000) Dual electroencephalogram markers of human sleep homeostasis: correlation between theta activity in waking and slow-wave activity in sleep. Neuroscience 101:523-529.
25. Stacey W, Le Van Quyen M, Mormann F, Schulze-Bonhage A (2011) What is the present-day EEG evidence for a preictal state? Epilepsy Res. 97:243-251.
26. Arieli A, Sterkin A, Grinvald A, Aertsen A (1996) Dynamics of ongoing activity: explanation of the large variability in evoked cortical responses. Science 273:1868-1871.27. Freestone D R, et al. (2013) A method for actively tracking excitability of brain net-works using a fully implantable monitoring system. Conf Proc IEEE Eng Med Biol Soc
27. Freestone D R, et al. (2013) A method for actively tracking excitability of brain net-works using a fully implantable monitoring system. Conf Proc IEEE Eng Med Biol Soc
28. Freestone D R, et al. (2011) Electrical probing of cortical excitability in patients with epilepsy. Epilepsy Behav 22 Suppl 1:S110-118.
29. Vyazovskiy V V, et al. (2011) Local sleep in awake rats. Nature 472:443-447.
30. Achermann P, Borb'ely A A (2011) Sleep homeostasis and models of sleep regulation

In Principles and Practice of Sleep Medicine (Elsevier Saunders), 5 th edition, pp 431-444.
31. Tononi G, Cirelli C (2003) Sleep and synaptic homeostasis: a hypothesis. Brain Res Bull 62:143-150.
32. Tononi G, Cirelli C (2006) Sleep function and synaptic homeostasis. Sleep Med Rev 10:49-62.
33. Bushey D, Tononi G, Cirelli C (2011) Sleep and synaptic homeostasis: structural evidence in drosophila. Science 332:1576-1581.
34. Cook M J, et al. (2013) Prediction of seizure likelihood with a long-term, implanted seizure advisory system in patients with drug-resistant epilepsy: a first-in-man study. Lancet Neurol 12:563-571.
35. Langton C G (1990) Computation at the edge of chaos: Phase transitions and emergent computation. Physica D 42:12-37.
36. Ortinski P, Meador K J (2004) Cognitive side effects of antiepileptic drugs. Epilepsy Behav 5 Suppl 1:S60-65.
37. Beggs J M, Plenz D (2003) Neuronal avalanches in neocortical circuits. J Neurosci

What is claimed is:

1. A method of continuously monitoring synchronization R in a subject comprising:
(a) determining a deviation in mean synchronization (R) from a predetermined value at rest, wherein the pre-determined value of R is 0.5, and the variability of synchronization H; and
(b) repeating step (a) one or more times to continuously monitor synchronization R and its variability H in a subject.

2. The method of claim 1, wherein step (a) comprises:
(i) continuously recording the electroencephalogram (EEG);
(ii) filtering the EEG;
(iii) calculating the instantaneous synchronization as a function of time across different channels in this frequency band;
(iv) calculating the mean synchronization R as the average of the instantaneous synchronization over time;
(v) calculating the variability of synchronization H;

3. The method of claim 2, wherein the EEG is continuously recorded at more than one site,

4. The method of claim 2, wherein the EEG is filtered between 50-100 Hz.

5. The method of claim 2, wherein EEG is recorded during multiple different recording sessions that can be several hours, several days, several weeks or years apart from each other. The method provides a record of these R and H values at all times when EEG was recorded and allows to display a history of all values in the past.

6. A method of determining the degree of brain excitability in a subject comprising:
(a) determining a deviation in mean synchronization (R) from a predetermined value at rest, wherein the pre-determined value of R is 0.5; and (b) repeating step (a) one or more times to continuously monitor synchronization R in a subject, wherein a change in R from the predetermined value indicates the degree of brain excitability in a subject.

7. A method of determining the degree of cognitive impairment in a subject comprising:
(a) determining a change in variability of synchronization H from a predetermined value at rest; and (b) repeating step (a) one or more times to continuously monitor variability of synchronization H in a subject, wherein a change in H from the pre-determined value indicates the degree of cognitive impairment in a subject.

8. A method of identifying subjects that are sleep deprived comprising:
(a) determining a deviation in mean synchronization (R) from a predetermined value at rest, wherein the pre-determined value of R is 0.5; and (b) repeating step (a) one or more times to continuously monitor synchronization R in a subject, wherein a change in R from the predetermined value indicates the degree of sleep deprivation in a subject.

9. The method of claim 1, wherein the subject is suffering from epilepsy.

10. The method of claim 1, wherein it is used as a biomarker for excitability.

11. The method of claim 1, wherein the effectiveness, function and therapeutic effect of one or more antiepileptic drugs is monitored.

12. The method of any one of claims 7-9, further comprising gathering data from other physiological sensors.

13. The method of any one of claim 1, or 7-9, wherein the method is operational with hardware or software or a combination thereof.
